(19) United States
(12) Patent Application Publication
(10) Pub. No.: US 2003/0192532 A1
(43) Pub. Date: Oct. 16, 2003

(54) Nebulizer

(76) Inventor: Andrew David Hopkins, Sutton Coldfield (GB)

Correspondence Address: KEUSEY, TUTUNJIAN & BITETTO, P.C., 14 VANDERVENTER AVENUE, SUITE 128, PORT WASHINGTON, NY, 11050, US

(21) Appl. No.: 10/121,237

(22) Filed: Apr. 12, 2002

(51) Int. Cl. A61M 11/00

(52) U.S. Cl. 128/200.14; 128/200.21

(57) ABSTRACT

The present invention relates to a nebulizer comprising an electrically energizable ultrasonic transducer having an oscillation frequency dependent current and voltage characteristic exhibiting the occurrence of a minimum magnitude of current through the transducer at an anti-resonance frequency of the transducer, the voltage across the transducer increasing with an increase in frequency within a range of frequencies including said anti-resonance frequency. The nebulizer further comprises a transducer drive system for generating a drive signal for energising the transducer, the drive system comprising oscillation frequency control means operable to maintain the frequency of the drive signal at a specific frequency within said range of frequencies by reference to the variation of both current through the transducer and voltage across the transducer with a change of frequency within the said frequency range. The transducer is arranged to receive said drive signal and to cause physical vibrations in fluid to be nebulized when energised by said drive signal. A transducer having a simple automatic frequency tuning system is thereby provided.

### FIELD OF THE INVENTION

The present invention relates to a nebulizer and, particularly, but not exclusively, to an ultrasonic nebulizer suitable for nebulizing/atomizing liquid based medication for the purpose of inhalation by a patient.

### BACKGROUND OF THE INVENTION

It is well known for liquid nebulizers to employ ultrasonic technology to provide a required degree of liquid atomization. Conventional ultrasonic nebulizers incorporate a piezo-oscillator element (i.e. a piezo-electric crystal) which is exposed to a liquid to be atomized and selectively excited by means of an electric oscillating circuit. Upon excitation, the piezo-oscillator element acts as a transducer and generates ultrasonic waves in the liquid, which are directed to the liquid surface. As a consequence, the surface of said liquid is atomized into a fine mist, which may be transported, as necessary, by suitable means such as an electric fan or simply by the inhalation of a patient.

The characteristics of piezo-oscillator elements are well known and must be carefully considered when designing the oscillating circuit of a nebulizer if efficient liquid atomization is to be achieved. Both the electrical and mist generating characteristics of a typical piezo-oscillator element are schematically shown in FIGS. 1a, 1b and 1c of the accompanying drawings. It will be seen by reference to FIG. 1a that the effective reactance of a piezo-oscillator element is inductive (i.e. positive) at frequencies between a series (resonance) frequency fᵣ and a parallel (anti-resonance) frequency fₐ of the transducer. At other frequencies, the effective reactance of the piezo-oscillator element is capacitive (i.e. negative). Also, it will be seen from FIG. 1b that the effective impedance of a piezo-oscillator element is minimized at the resonance frequency fᵣ and maximized at the anti-resonance frequency fₐ.

The oscillation amplitude of a piezo-oscillator element is maximized at the resonance frequency fᵣ, however it will be seen from FIG. 1c that this frequency fᵣ is somewhat less than the optimum frequency fₒₘ for maximizing liquid atomization. Indeed, the optimum frequency fₒₘ for maximum yield of mist is between the resonance frequency fᵣ and the anti-resonance frequency fₐ.

Notwithstanding the fact that the optimum frequency in terms of mist generation is between the resonance and anti-resonance frequencies, the majority of conventional ultrasonic nebulizers incorporate oscillating circuitry, which uses one of these frequencies as a basis for exciting the piezo-oscillating element. Typically, the oscillating circuitry is designed to automatically tune to one of said frequencies so that variations in, for example, ambient conditions, may be conveniently accommodated. A number of methods are employed for achieving this automatic tuning, one of which uses the voltage and current phases in respect of a piezo-oscillating element in conjunction with a phase comparator (see, for example, U.S. Pat. No. 3,819,961). However, a problem associated with the prior art systems for controlling the oscillation frequency of piezo-oscillating elements is that such systems are generally relatively complicated.

In order for a medication dispensed by a nebulizer to be adequately received by a patient, it is important for the atomized liquid particles to have an appropriate size. Release of undesirably large particles is prevented in many prior art nebulizers by means of a baffle system. However, the effectiveness of such systems is heavily dependent upon fluid flow velocity and the resultant impinging of particles on one or more baffles tends to leave a large proportion of medication, which is not inhaled. This reduces the overall effectiveness and efficiency of prior art nebulizers.

### SUMMARY

It is an object of the present invention to provide an ultrasonic nebulizer comprising means for automatically tuning to a required oscillation frequency wherein said means is comparatively simple and may be readily and inexpensively manufactured.

It is also an object of the present invention to provide a nebulizer incorporating effective and efficient means for controlling the size of atomized particles.

A first aspect of the present invention provides a nebulizer comprising an electrically energizable ultrasonic transducer having an oscillation frequency dependent current and voltage characteristic exhibiting the occurrence of a minimum magnitude of current through the transducer at an anti-resonance frequency of the transducer, the voltage across the transducer increasing with an increase in frequency within a range of frequencies including said anti-resonance frequency; a transducer drive system for generating a drive signal for energizing the transducer, the drive system comprising oscillation frequency control means operable to maintain the frequency of the drive signal at a specific frequency within said range of frequencies by reference to the variation of both current through the transducer and voltage across the transducer with a change of frequency within the said frequency range, the transducer being arranged to receive said drive signal and to cause physical vibrations in the fluid to be nebulized when energized by said drive signal.

It is preferable for the oscillation frequency control means to maintain the frequency of the drive signal at a magnitude at which the variation with frequency of a function of current through the transducer is zero. It is also preferable for the function of current to have a zero variation with frequency at a frequency magnitude other than the anti-resonance frequency of the transducer. The function of current may have a zero variation with frequency at a frequency magnitude at which the yield of fluid nebulized by the transducer is substantially maximised.

The oscillation frequency control means may comprise a comparator for determining the direction of change with frequency of the function of current through the transducer. Furthermore, the oscillation frequency control means may also comprise a comparator for determining the direction of change with frequency of voltage across the transducer. Preferably, outputs from the two comparators are applied to an exclusive OR gate for determination of the frequency magnitude at which the function of current has a zero variation with frequency. The function of current may be such that the relationship of said function with frequency is identical to that of current through the transducer with frequency.

A second aspect of the present invention provides a nebulizer comprising means for inducing a swirling motion in nebulized fluid about a swirl axis as said nebulized fluid is extracted from the nebulizer, the angular velocity of the swirling fluid being sufficient to result in undesirably large nebulized particles moving in a direction offset to the direction of swirl and thereby away from the swirl axis. Thus, oversized nebulized/atomized particles are effectively thrown from the swirling fluid. These oversized particles maybe then collected and returned to a fluid reservoir.

The means for inducing a swirling motion in nebulized fluid preferably comprises one or more guides for deflecting a flow of fluid. The guide may be a curved guide or vane.

The nebulizer preferably comprises a first chamber, in which fluid is nebulized, and a second chamber having an inlet and outlet and in which the means for inducing a swirling motion is arranged so as to induce a swirling motion in fluid passing through the second chamber from said inlet to said outlet; the first and second chambers being in fluid communication so that, when in use, swirling fluid in the second chamber tends to draw nebulized fluid into said second chamber from the first chamber. The means by which the first and second chambers are in fluid communication preferably comprises a passage joining the first and second chambers wherein said passage is centered on the swirl axis. The passage may comprise an aperture in a wall common to both the first and second chambers. Means may be provided for allowing fluid to flow into the first chamber as nebulized fluid is drawn into the second chamber.

The nebulizer preferably comprises means for collecting undesirably large nebulized particles, which have moved away from the swirl axis. Said collection means preferably directs collected particles to the first chamber for re-nebulization.

The first chamber preferably comprises a tube located above the surface of liquid to be nebulized so that the interior of the tube receives the majority of any nebulized fluid. One or more slots may be provided in the wall of the tube so as to allow nebulized fluid to pass from the interior of the tube to the exterior thereof.

A third aspect of the present invention provides a nebulizer comprising a demand prediction system which determines the period of a cyclical demand for nebulized fluid by monitoring fluid flow with detection means so as to determine the start of consecutive demand cycles and compensating for lag between the actual start of a demand cycle and the detected start of said cycle by calculating the start of a next demand cycle as being a length of time from the detected start of a current demand cycle where said length of time is equal to the determined period less a fixed length of time.

Said fixed length of time may be equal to or greater than the length of time between the actual start of a demand cycle and the detected start of said cycle. The detection means preferably comprises a thermistor.

### BRIEF DESCRIPTION OF THE DRAWINGS

An embodiment of the present invention will now be described with reference to the accompanying drawings, in which:

FIGS. 1a, 1b and 1c respectively show the variation with frequency of reactance, impedance and quality of mist generation of a piezo-oscillator element;

FIG. 2 shows a schematic perspective view of a nebulizer according to the present invention;

FIG. 3 shows a schematic partial perspective view of the nebulizer shown in FIG. 2;

FIG. 4 shows graphically the basis for operation of a breath prediction system;

FIG. 5 shows the variation with frequency of voltage across a transducer (i.e. a piezo-oscillator element) and of current through a transducer;

FIG. 6 is a block diagram showing the structure of the electric circuit of the nebulizer shown in FIG. 2;

FIG. 7 is a diagram of the control circuitry of the nebulizer shown in FIG. 2;

FIG. 8 is a diagram of the power supply circuitry of the nebulizer shown in FIG. 2; and

FIG. 9 is a block diagram showing the interface between the control circuitry of FIG. 7 and the power supply circuitry of FIG. 8.

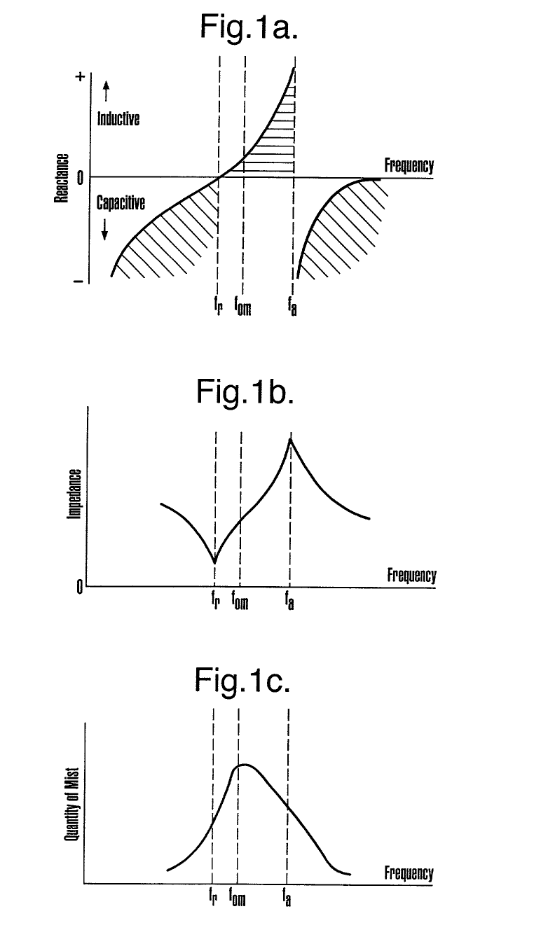

### DETAILED DESCRIPTION OF THE INVENTION

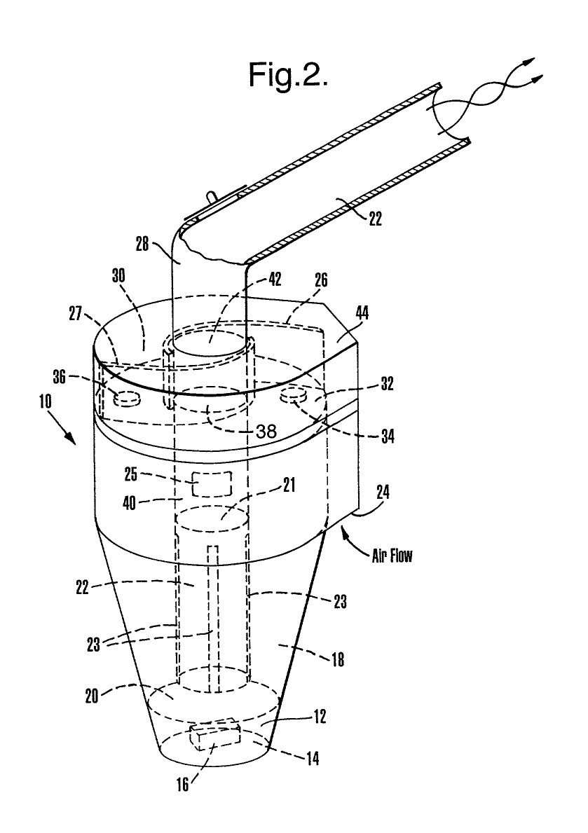

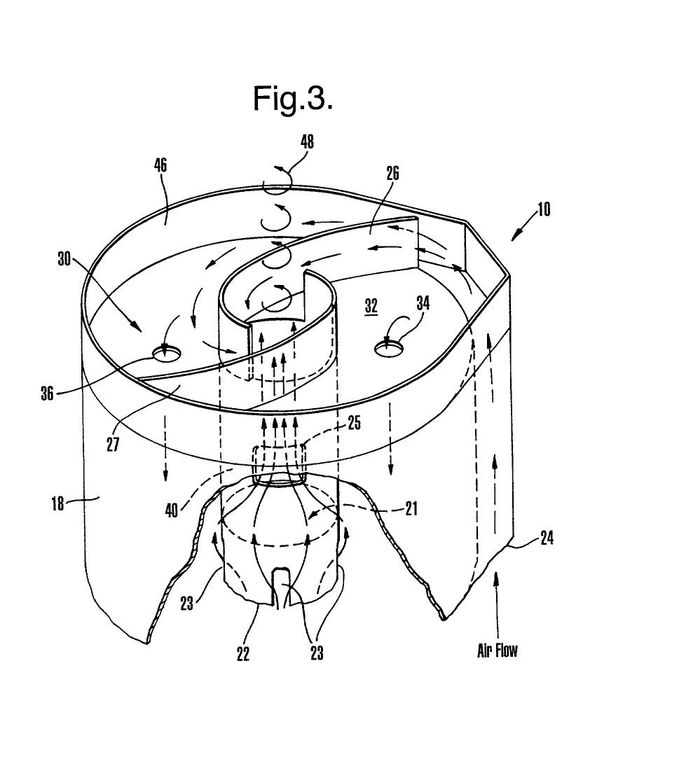

A nebulizer 10 according to the present invention incorporates a reservoir 12 for retaining a liquid based medication 14 to be nebulized (see FIGS. 2 and 3). This liquid 14 may include a solution or suspension of solid compounds. As is known in the art, an ultrasonic transducer 16 (i.e. a piezo-oscillator element) is mounted relative to the reservoir 12 so as to be exposed to the liquid medication 14. The transducer 16 is itself connected to appropriate electrical control circuitry (not shown in FIGS. 2 and 3) for providing excitation energy at a required frequency. The control circuitry is powered by means of rechargeable batteries.

A nebulizing chamber 18 is located above the surface 20 of the liquid medication 14 so as to receive liquid, which has been atomized. A mouthpiece 22 extends into the nebulizing chamber 18 to allow a user of the nebulizer 10 (i.e. a patient) to inhale any atomized medication produced following excitation of the transducer 16. The lower end of the mouthpiece 22 is located adjacent the transducer 16 so as to receive all (or a substantial proportion) of the atomized medication generated during excitation. Atomized mediation is generally ejected from the surface of the liquid 14 as a fountain of small particles. In order to prevent these particles from being undesirably ejected from the nebulizer 10 through the mouthpiece 22, a barrier member 21 is located in the mouthpiece 22. The barrier member 21 is in the form of a concave dome and completely blocks the passage of fluid through the mouthpiece 22. In use, atomized medication to be inhaled by a patient bypasses the barrier member 21 by flowing from a location in the mouthpiece 22 below said member 21 into the nebulizing chamber 18 via one of four elongate slots 23. The slots 23 are equi-spaced about the circumference of the mouthpiece 22. The atomized medication then flows through one of two apertures 25 to a location in the mouthpiece 22 above the barrier member 21. The two apertures 25 are located on opposite sides of the mouthpiece 22.

An inlet port 24 (provided as necessary with an appropriate one way valve) ensures that fluid pressure within the nebulizer 10 is equalised with the atmospheric pressure external to the nebulizer 10 as atomized medication is inhaled.

The nebulizer 10 further incorporates breath actuation means (not shown in FIGS. 2 and 3) in the form of electric circuitry, which ensures that atomized medication is generated only as and when it is required by the user. In this way, the components of the nebulizer 10 are not used unnecessarily and, accordingly, the efficiency of the atomization process is improved and the service life of the nebulizer 10 is extended. The breath actuation means of the present embodiment incorporates a heated thermistor exposed to the flow path of atomized liquid drawn from the nebulizing chamber 18. As a fluid flow is initiated upon the inhalation of a patient, the heated thermistor is cooled. This produces a change in thermistor resistance. Excitation of the transducer 16 may be thereby triggered.

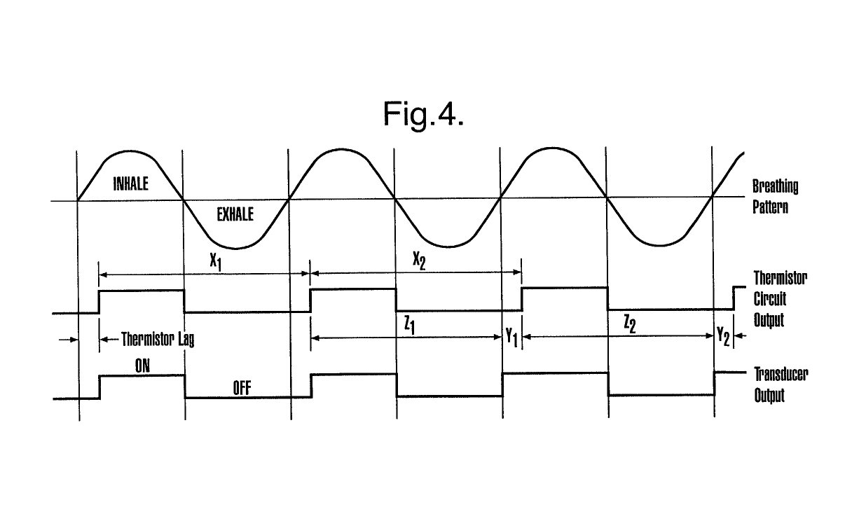

Due to the thermal lag of a thermistor, there is a small delay between the start of inhalation and the detection of a breath. Also, once a transducer is excited, there is a small delay before atomized medication is generated. Accordingly, it is preferable for the breath actuation means of the nebulizer 10 to be turned on for a fixed pre-determined period before actual detection of a breath. With reference to FIG. 4, it will be seen that this is achieved by measuring the period X of a user's previous breathing cycle and using this measured time, less the fixed pre-determined delay time Y, to turn on the nebulizer after a calculated period Z (wherein Z=X−Y) from the detected start of the present breath. This is repeated continually for each breath and, for a series of breaths having the same period, results in the nebulizer being turned on at the same as the user begins to inhale. It will however be appreciated that in one embodiment the first two breaths of the user allow a calculation of the breath period X and, accordingly, anticipation of the user's inhalations is only made for the user's third and subsequent breaths. The continual monitoring of each breath allows adjustments to be made for variations in the user's rate of breathing. In other words, the system allows for variations in the breath period X. Also, by increasing the pre-determined delay time Y, the system maybe configured to initiate excitation of the transducer slightly before the anticipated inhalation of a user so as to pre-fill the nebulizing chamber 18 with atomized medication. This results in a faster treatment time. Furthermore, because of the characteristics of a thermistor as a flow sensor, nebulization maybe terminated before the end of inhalation so as to reduce the amount of medication being re-breathed into the atmosphere. This demand prediction system is preferably implemented by means of appropriate electronic circuitry.

The breath actuation means may be designed so that, once initiated, atomization continues until the heated thermistor senses a temperature change indicating the end of patient inhalation. Alternatively, the breath actuation means may be designed so that atomization is terminated when the heated thermistor senses a decrease in breathing rate to, for example, 80% of the maximum breathing rate detected during the current breath. In yet further designs of breath actuation means, atomization may be terminated after a fixed pre-determined time period or after a period dependent upon the length of the previous breath. Termination of atomization may also be made dependent upon the period of time taken for the thermistor to detect a pre-determined change in the peak value measured during the current breath or the average value of the previous breaths, wherein said pre-determined change is a pre-determined percentage of said peak value. Breath actuation means employing a heated thermistor to detect fluid flow is disclosed in EP 0 178 925 A2.

The present embodiment also incorporates means for detecting when the reservoir 12 has been emptied (or is about to be emptied) of the liquid based medication 14 and for switching off the nebulizer 10 upon such detection and indicating to the user that the source of medication has been exhausted (or is about to be exhausted). The means for detecting exhaustion of the medication source incorporates control logic, which compares the current passing through the transducer with pre-determined values of current magnitude corresponding to a minimum acceptable level of liquid medication.

As the reservoir 12 is emptied, the transducer 16 becomes unloaded and the current passing therethrough reduces. Accordingly, the control logic can be conveniently designed to switch off the nebulizer once the magnitude of the transducer current has dropped to the predetermined level. At this stage, an LED (i.e. a Light Emitting Diode) or similar device may be activated by said control logic so as to provide the required indication to the user. Appropriate means for detecting a minimum acceptable level of liquid within the reservoir 12 is disclosed in GB 2,219,512 A.

In order for the medication dispensed by the nebulizer 10 to be adequately received by a patient, it is important that the particles of the atomized liquid have an appropriate size. Ideally, the diameter of the particles should be between 0.5 :m and 5.0 :m. Accordingly, as in many prior art nebulizer designs, the present embodiment is provided with a transducer capable of generating maximum quantities of mist in a frequency range of approximately 1 to 2.5 MHz. This frequency range produces particles of the desired size. In addition, many prior art nebulizers limit particle size by means of a baffle system. However, the effectiveness of such systems is heavily dependent upon fluid flow velocity and the resultant impinging of particles on one or more baffles tends to result in a large proportion of medication (i.e. residual medication) not being inhaled by the patient.

In order to overcome this problem, the present nebulizer 10 incorporates two vanes 26,27 (although a single vane may be sufficient) for generating fluid swirl in an upper portion 28 of the mouthpiece 22. The two vanes 26,27 are located in an upper chamber 30 situated above the nebulizing chamber 18. A wall 32 common to both the upper chamber 30 and the nebulizing chamber 18 is provided with two apertures 34,36 which, in use, permit air to flow into the nebulizing chamber 18 from the upper chamber 30.

A central aperture 38 is also provided in the wall 32 for permitting a flow of air and atomized medication to pass from the nebulizing chamber 18 into the upper chamber 30 via a lower portion 40 of the mouthpiece 22. A second central aperture 42 is defined opposite said first central aperture 38 in an upper wall 44 (not shown in FIG. 3) of the nebulizer 10. The upper portion 28 of the mouthpiece 22 extends from the second central aperture 42 outwardly from the upper chamber 30. The mouthpiece 22 does not extend through the upper chamber 30. Furthermore, the two vanes 26,27 are arranged to generate a swirling flow of fluid in the region between the two central apertures 38,42.

The arrangement is such that, in use, inhalation by a patient through the upper portion 28 of the mouthpiece 22 causes air to enter the nebulizer 10 at the inlet port 24. By reference to FIG. 3, it will be seen that the intake of air progresses into the upper chamber 30 where it is separated into two distinct flows by a first vane 26. The vanes 26,27 and a curved sidewall 46 of the upper chamber 30 are such that the separated airflows are directed to the centre of the upper chamber 30 between the two central apertures 38,42. The airflows are so directed that a swirling motion is induced therein.

Vortices 48 are formed in the region of the central apertures 38,42 which, due to their lower static fluid pressure, tend to draw a mixture of air and atomized medication from the nebulizing chamber 18 into the upper chamber 30 via the lower portion 40 of the mouthpiece 22. This fluid flow from the nebulizing chamber 18 results in a flow of air into the nebulizing chamber 18 from the upper chamber 30 via the apertures 34,36. The vortices 48 and atomized medication drawn into the upper chamber 30 are inhaled by the patient via the upper portion 28 of the mouthpiece 22.

The spinning fluid flow rotates with sufficient velocity for undesirably large particles of atomized medication to be effectively thrown against the vanes 26,27 (and/or to the sides of the mouthpiece 22). Said large particles of medication are then received in a skirt or gutter (not shown) and thereby directed back to the reservoir 12 under the action of gravity. In this way, the amount of residual medication arising through use of the nebulizer 10 is minimized. In one embodiment, at least one vane may include a radius of curvature, which decreases in a direction towards the swirl axis.

With regard to the control of residual medication, the mouthpiece of an alternative embodiment may be provided as two concentrically mounted tubes wherein atomized medication is inhaled by a patient through the inner tube while pressure equalization within the nebulizer is achieved by virtue of an air flow into the nebulizer which passes through a generally annular shaped passage formed between said tubes. Thus, large particles leaving the nebulizer chamber tend to become entrained in the incoming airflow. This further obviates the need for an internal baffle system.

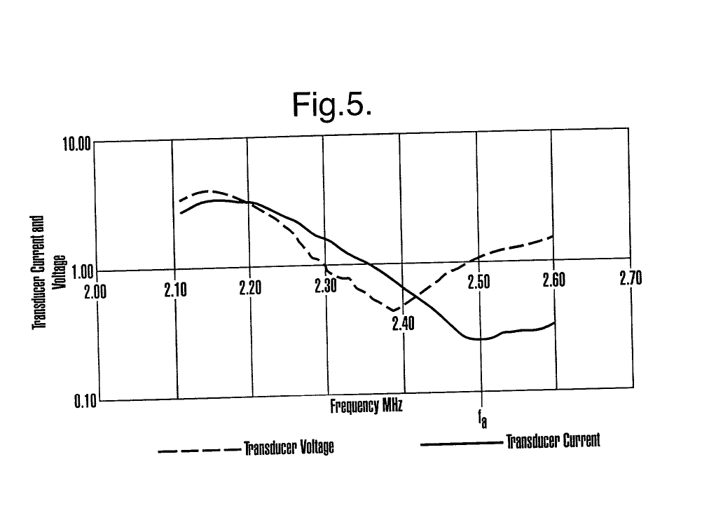

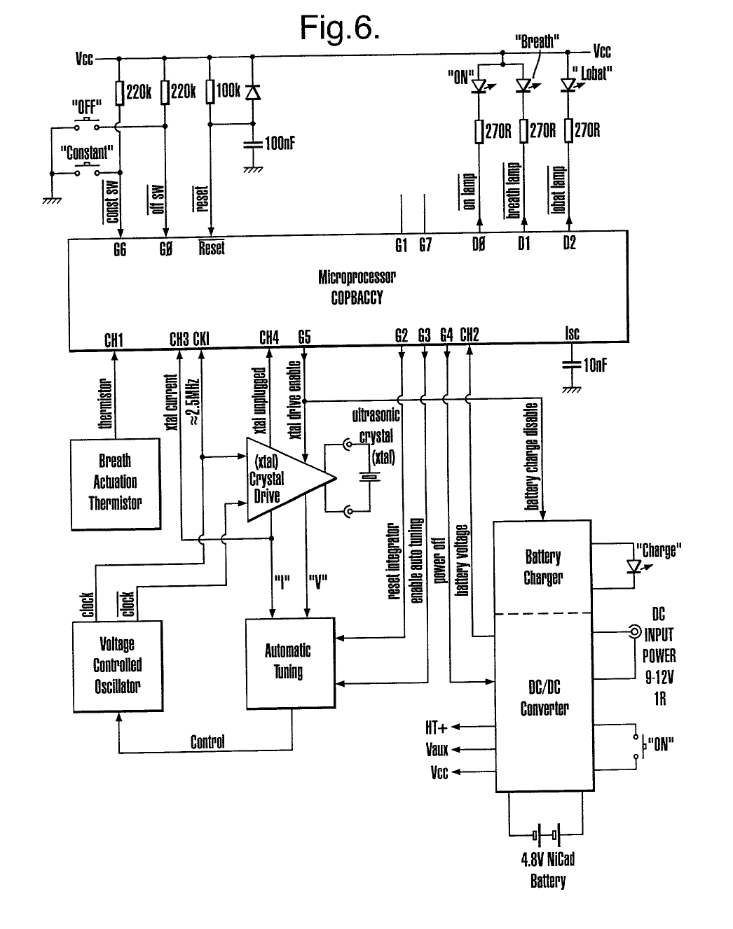

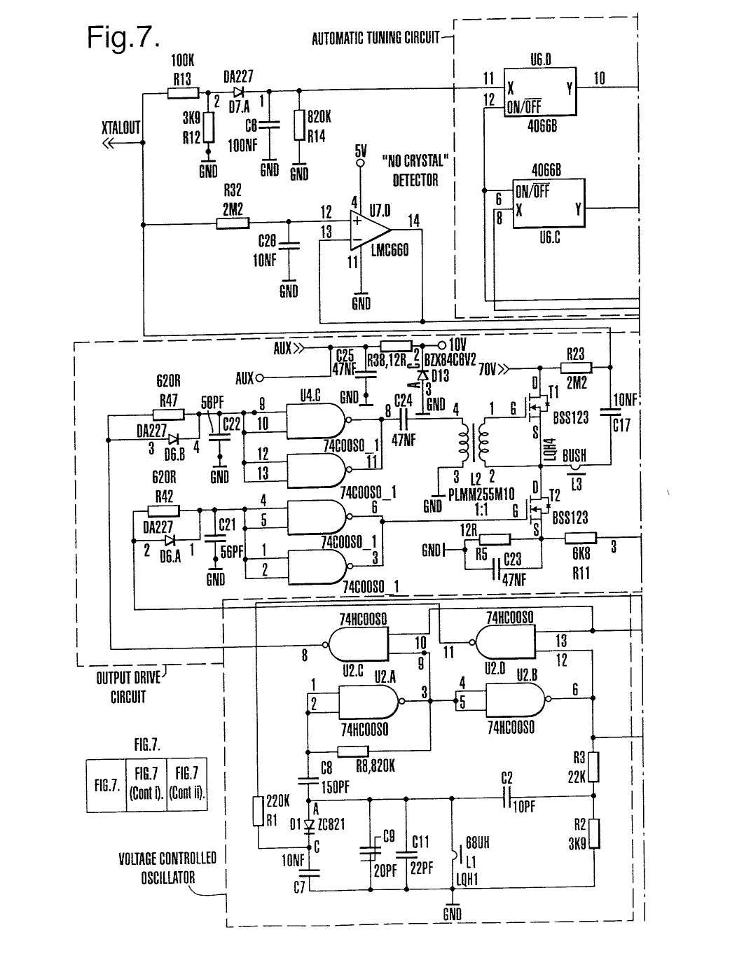

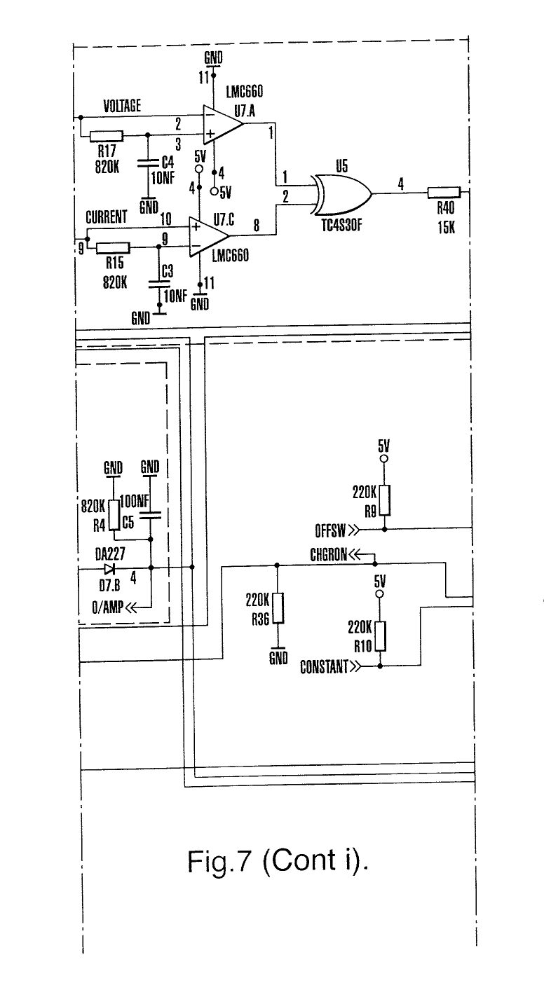

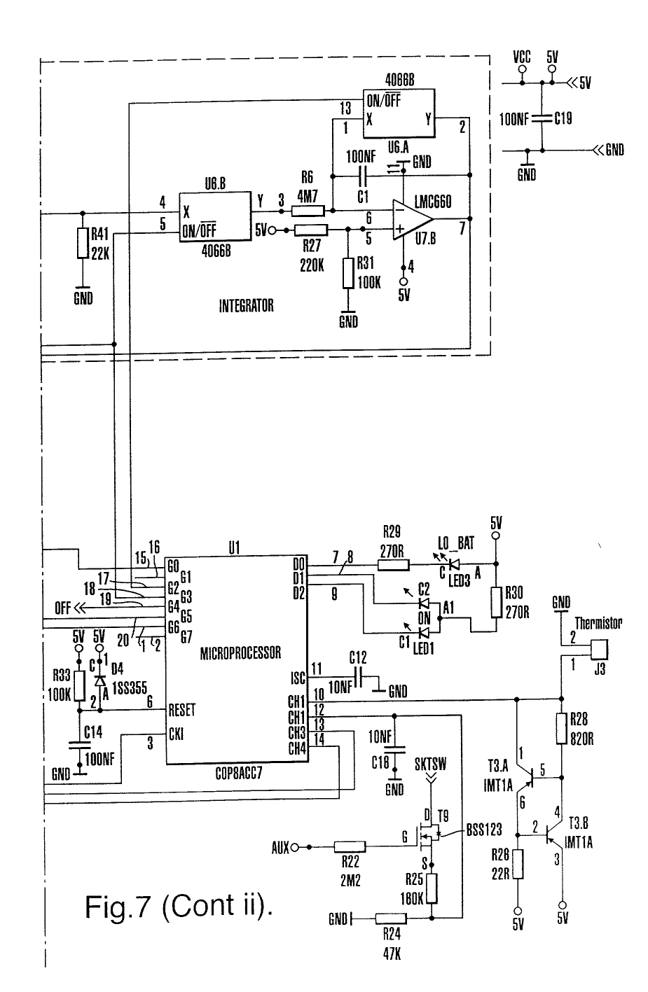

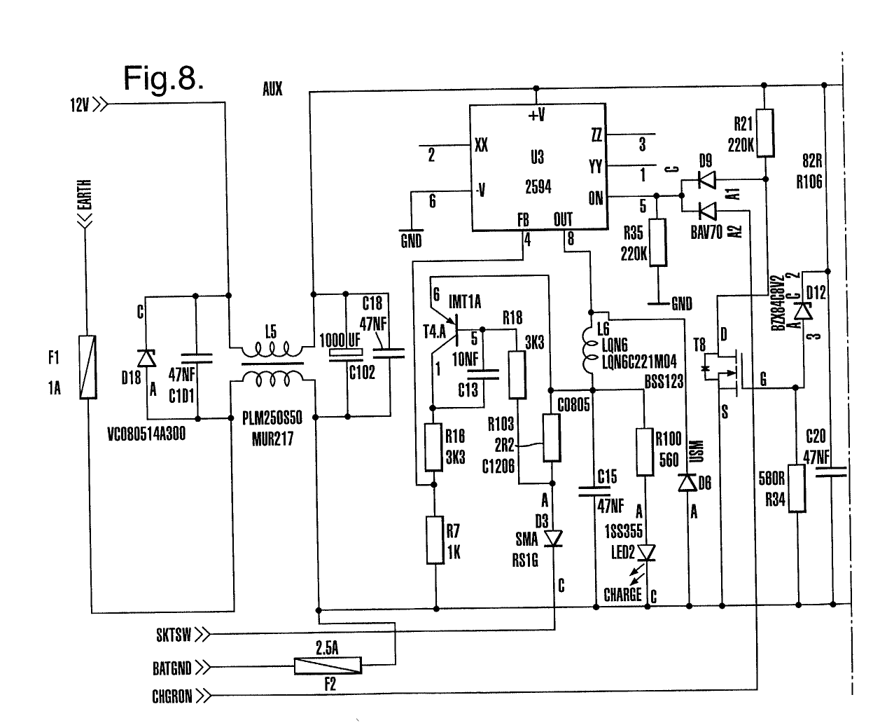

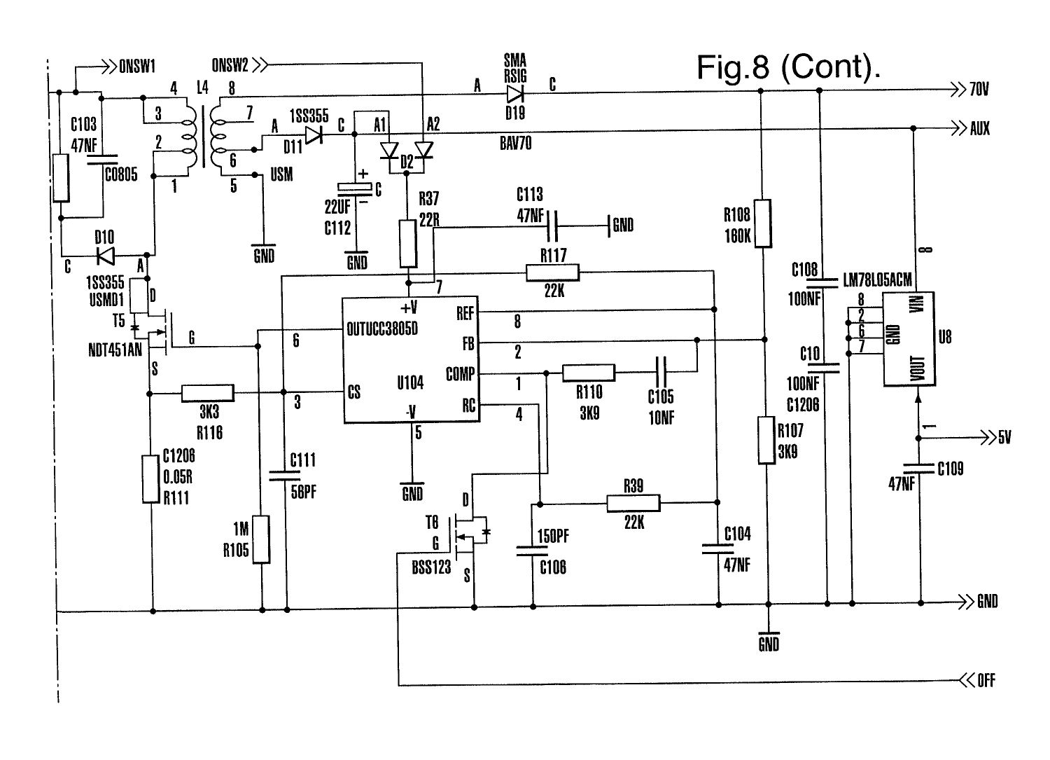

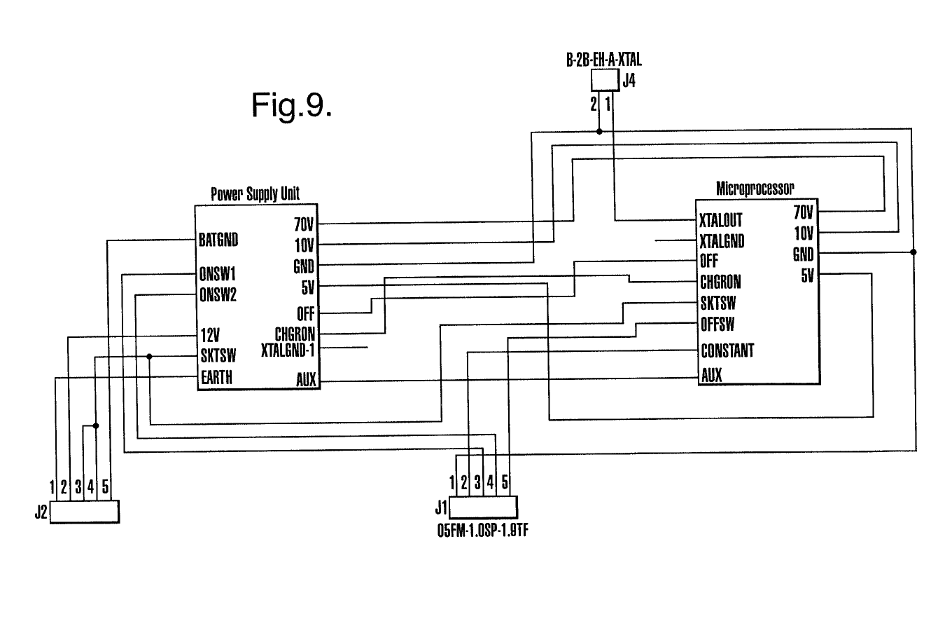

The control and power supply circuits of the present embodiment are illustratively shown in FIGS. 7 and 8 respectively. The relationship between these two circuits is illustratively shown in FIG. 9. The general structure of the nebulizer circuitry is illustratively shown in FIG. 6. An automatic tuning circuit (see FIG. 7) is comprised in the control circuitry for automatically tuning transducer oscillation to a specific frequency. This tuning circuit operates on the basis of the transducer current/voltage and frequency relationship shown in FIG. 5.

With reference to FIG. 5, it will be seen that the current passing through a transducer varies with frequency and falls to a minimum at the parallel (i.e. anti-resonance) frequency fₐ of the transducer. In addition, it will be seen that the voltage across the transducer at frequencies in the region of the parallel frequency fₐ increases with frequency. This current/voltage and frequency relationship is characteristic of ultrasonic transducers and is used in the present nebulizer to provide directional control to the automatic tuning circuit.

The aforementioned transducer characteristic may be conveniently used to automatically tune to the anti-resonance frequency. The tuning circuit comprises two comparators for determining the direction of change (i.e. increase or decrease) of either transducer voltage or current with frequency. One of the comparators is presented with a voltage representing the magnitude of the current through the transducer, while the other comparator is presented with a voltage representing the magnitude of the voltage across the transducer. As will be apparent to those skilled in the art, a part of the signal applied to each comparator is delayed for comparison with the instant signal. It may then be determined whether or not the applied signal is momentarily rising or falling. It will be seen that the output of the two comparators is applied to an exclusive OR gate in order to determine whether the transducer frequency is greater than or less than the frequency at which the transducer current is a minimum (i.e. the anti-resonance frequency fₐ). On the basis of this determination, the frequency may be appropriately adjusted.

The exclusive OR gate produces a clock output of varying pulse widths which is averaged with an integrator (see FIG. 7) so as to produce a signal for driving a voltage controlled oscillator. This oscillator is operable at frequencies of approximately 2.5 MHz so as to ensure that, as previously discussed, the atomized liquid is generated with the desired particle size. It should be understood that the magnitudes and placement of circuit components are shown for illustrative purposes and should not be construed as limiting the present invention. The magnitudes and components may be adjusted and changed by one skilled in the art in view of the present disclosure.

Two anti-phase clocks are used to operate an output drive circuit (see FIG. 7). It will be seen that the output drive circuit comprises two field-effect transistors (i.e. two FETs) arranged in a totem-pole configuration so as to allow the piezo-electric transducer of the nebulizer to be referenced to ground. A level-shifting network comprising a small transformer is provided in the output drive circuit for stepping up the circuit voltage to between 40V and 100V. This higher voltage is used to drive the upper FET. With appropriate signal rectification, the illustrated output drive circuit and automatic tuning circuit generate a measured rectified and smoothed transducer voltage/current and tune the oscillator to the required anti-resonance frequency.

In alternative embodiments, the circuitry of the nebulizer may be configured so as to automatically tune to a frequency, which is offset from the anti-resonance frequency fₐ. This is achieved by manipulating the transducer voltage and current magnitudes so as to generate a frequency curve having a maximum or minimum occurring at a frequency other than that at which the minimum current through the transducer is found. This new maximum or minimum is then used as a new reference for the automatic tuning. For example, the tuning frequency can be offset by reference to the modulus of resistance (i.e. V/I) of the transducer. This is found by dividing the measured rectified and smoothed voltage across the transducer by the measured rectified and smoothed current through the transducer. A curve is thereby produced which has a maximum occurring at a frequency greater than the frequency at which the minimum current occurs (i.e. the anti-resonance frequency fₐ). The V/I function is derived in practice by a divider circuit. As an alternative to dividing the transducer voltage by the transducer current, these voltage and current variables may be either added (V+I), subtracted (V−I) or multiplied (V×I) so as to generate an offsetting of frequency from the anti-resonance frequency.

The present invention is not limited to the specific embodiments or methods described above. Alternative arrangements may be apparent to a reader skilled in the art.

1. A nebulizer comprising:
an electrically energizable ultrasonic transducer having an oscillation frequency dependent current and voltage characteristic exhibiting the occurrence of a minimum magnitude of current through the transducer at an anti-resonance frequency of the transducer, the voltage across the transducer increasing with an increase in frequency within a range of frequencies including said anti-resonance frequency;
a transducer drive system for generating a drive signal for energizing the transducer, the drive system comprising oscillation frequency control means operable to maintain the frequency of the drive signal at a specific frequency within said range of frequencies by reference to the variation of both current through the transducer and voltage across the transducer with a change of frequency within the said frequency range, the transducer being arranged to receive said drive signal and to cause physical vibrations in fluid to be nebulized when energized by said drive signal.

2. A nebulizer as claimed in claim 1, wherein the oscillation frequency control means maintains the frequency of the drive signal at a magnitude at which the variation with frequency of a function of current through the transducer is zero.

3. A nebulizer as claimed in claim 2, wherein the function of current is such that the relationship of said function with frequency is identical to that of current through the transducer with frequency.

4. A nebulizer as claimed in claim 2, wherein the function of current has a zero variation with frequency at a frequency magnitude other than the anti-resonance frequency of the transducer.

5. A nebulizer as claimed in claim 4, wherein the function of current has a zero variation with frequency at a frequency magnitude at which the yield of fluid nebulized by the transducer is substantially maximized.

6. A nebulizer as claimed in claim 1, wherein the oscillation frequency control means comprises a comparator for determining the direction of change with frequency of the function of current through the transducer.

7. A nebulizer as claimed in claim 6, wherein the oscillation frequency control means comprises a comparator for determining the direction of change with frequency of voltage across the transducer.

8. A nebulizer as claimed in claim 7, wherein outputs from the two comparators are applied to an exclusive OR gate for determination of the frequency magnitude at which the function of current has a zero variation with frequency.

9. A nebulizer comprising means for inducing a swirling motion in nebulized fluid about a swirl axis as said nebulized fluid is extracted from the nebulizer, the angular velocity of the swirling fluid being sufficient to result in undesirably large nebulized particles moving in a direction offset to the direction of swirl and thereby away from the swirl axis.

10. A nebulizer as claimed in claim 9, wherein the means for inducing includes at least one vane.

11. A nebulizer as claimed in claim 9, wherein the at least one vane includes a radius of curvature which decreases in a direction towards the swirl axis.

12. A nebulizer comprising a demand prediction system which determines a period of a cyclical demand for nebulized fluid by monitoring fluid flow with detection means so as to determine the start of consecutive demand cycles and compensating for lag between the actual start of a demand cycle and the detected start of said cycle by calculating the start of a next demand cycle as being a length of time from the detected start of a current demand cycle where said length of time is equal to the determined period less a fixed length of time.

13. A nebulizer as claimed in claim 12, wherein the system includes means for determining when demand has ceased.

14. A nebulizer as claimed in claim 13, wherein the means for determining includes a calculation based on breathing rate.
